(19) United States
(12) Patent Application Publication
(10) Pub. No.: US 2002/0032226 A1
(43) Pub. Date: Mar. 14, 2002

(54) Novel form of an oxazole compound

(76) Inventors: Thomas W. Schultz, Richboro, PA (US); Daniel Korey, Yardley, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/866,362

(22) Filed: May 25, 2001

Related U.S. Application Data
(60) Provisional application No. 60/207,179, filed on May 26, 2000.

(51) Int. Cl. A61K 31/421; C07D 263/30

(52) U.S. Cl. 514/379; 548/235

(57) ABSTRACT

This invention is directed to an amorphous 5-(4-aminosulfonyl-3-fluorophenyl)-4-cyclohexyl-2-methyloxazole compound, substantially free of crystals, methods for preparing the compound and pharmaceutical compositions containing the compound.

### FIELD OF THE INVENTION

The present invention is directed to a novel amorphous oxazole compound and a method for the preparation thereof. More particularly, this invention is directed to an amorphous 5-(4-aminosulfonyl-3-fluorophenyl)-4-cyclohexyl-2-methyloxazole compound, substantially free of crystals, having an improved dissolution rate over crystalline forms of the compound, methods of preparing the compound and pharmaceutical compositions containing the compound.

### BACKGROUND OF THE INVENTION

Arachidonic acid metabolites such as prostaglandin E₂ (PGE₂), prostaglandin G₂ (PGG₂), prostaglandin H₂ (PGH₂), prostaglandin I₂ (PGI₂) and thromboxane B₂ (TXB₂) play major roles in the inflammation process. A number of selective COX-2 inhibitors that prevent prostaglandin production have been described as useful in the treatment of cyclooxygenase-2 (COX-2) mediated disorders.

U.S. Pat. No. 5,994,381 to Haruta, et al., hereby incorporated by reference, describes a group of heteroaromatic oxazole compounds as specific COX-2 inhibitors having the general formula:

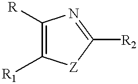

wherein Z is an oxygen atom, R₂ is a lower alkyl or a halogenated lower alkyl and one of R and R₁ is a group of the formula:

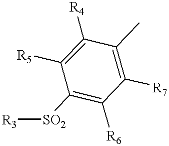

wherein R₃ is lower alkyl, amino or lower alkylamino, and R₄, R₅, R₆ and R₇ are the same or different and each is hydrogen atom, halogen atom, lower alkyl, lower alkoxy, trifluoromethyl, hydroxy or amino; provided that at least one of R₄, R₅, R₆ and R₇ is not hydrogen atom and the other is an optionally substituted cycloalkyl, an optionally substituted heterocyclic group or an optionally substituted aryl and a pharmaceutically acceptable salt thereof.

Also described by the Haruta '381 patent is the compound 5-(4-aminosulfonyl-3-fluorophenyl)-4-cyclohexyl-2-methyloxazole, having the formula:

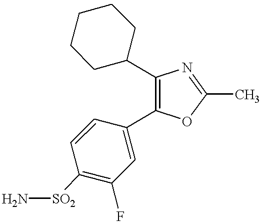

Prior references, however, do not disclose an amorphous 5-(4-aminosulfonyl-3-fluorophenyl)-4-cyclohexyl-2-methyloxazole compound, substantially free of crystals.

An object of the present invention is to provide a novel amorphous oxazole compound. Another object of the present invention is to provide an amorphous 5-(4-aminosulfonyl-3-fluorophenyl)-4-cyclohexyl-2-methyloxazole compound, substantially free of crystals, having an improved dissolution rate over a crystalline compound. Still another object of the present invention is to provide a pharmaceutical composition comprising an amorphous 5-(4-aminosulfonyl-3-fluorophenyl)-4-cyclohexyl-2-methyloxazole compound, substantially free of crystals, and a pharmaceutically acceptable carrier. A further object of the present invention is to provide a method for the preparation of an amorphous 5-(4-aminosulfonyl-3-fluorophenyl)-4-cyclohexyl-2-methyloxazole compound, substantially free of crystals.

### BRIEF DESCRIPTION OF THE DRAWINGS

FIG. 1 shows an X-ray powder diffraction (XRPD) pattern for an amorphous 5-(4-aminosulfonyl-3-fluorophenyl)-4-cyclohexyl-2-methyloxazole compound, substantially free of crystals, graphing Intensity (in counts/sec) against Angle 2Θ (in degrees).

FIG. 2 shows XRPD patterns for a crystalline 5-(4-aminosulfonyl-3-fluorophenyl)-4-cyclohexyl-2-methyloxazole compound, having Form A and Form B, graphing Intensity (in counts/sec) against Angle 2Θ (in degrees).

FIG. 3 shows a differential scanning calorimetry (DSC) thermogram for the amorphous compound, substantially free of crystals, graphing Heat Flow (in 20 MilliWatt) against Temperature-Time (° C.-min).

FIG. 4 shows the dissolution rate in water for the amorphous compound and the crystalline compound, having Form B, graphing Concentration (μg/mL) over Time (min).

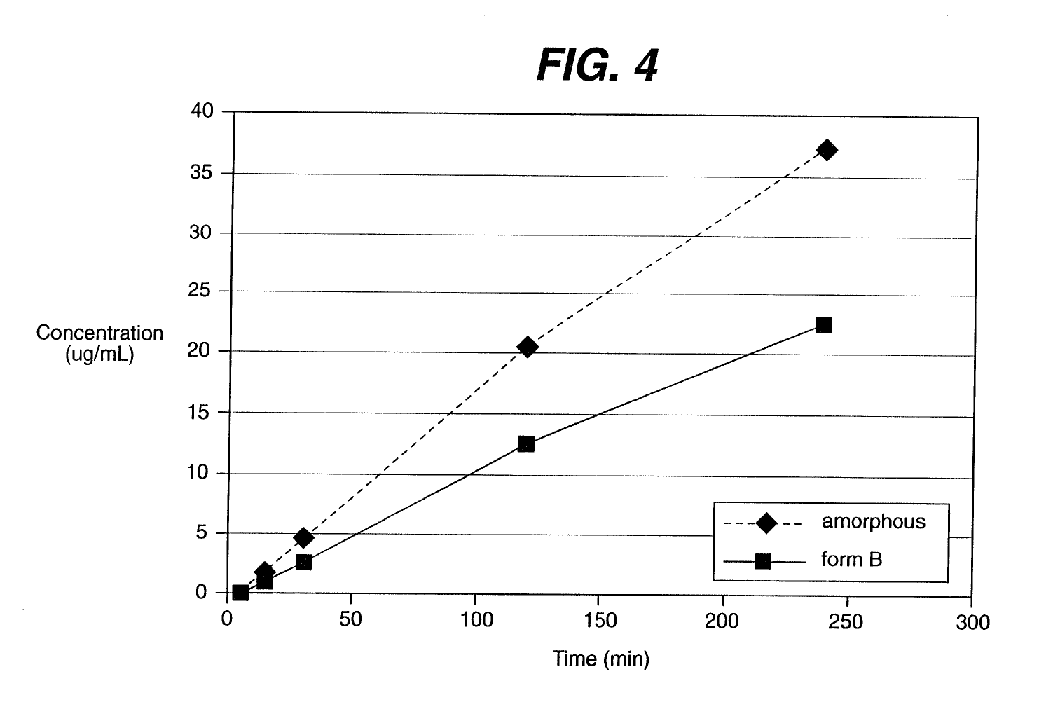

FIG. 5 shows the dissolution rate in simulated intestinal fluid (SIF) (minus pancreatin) for the amorphous compound and the crystalline compound, having Form B, graphing Concentration (μg/mL) over Time (min).

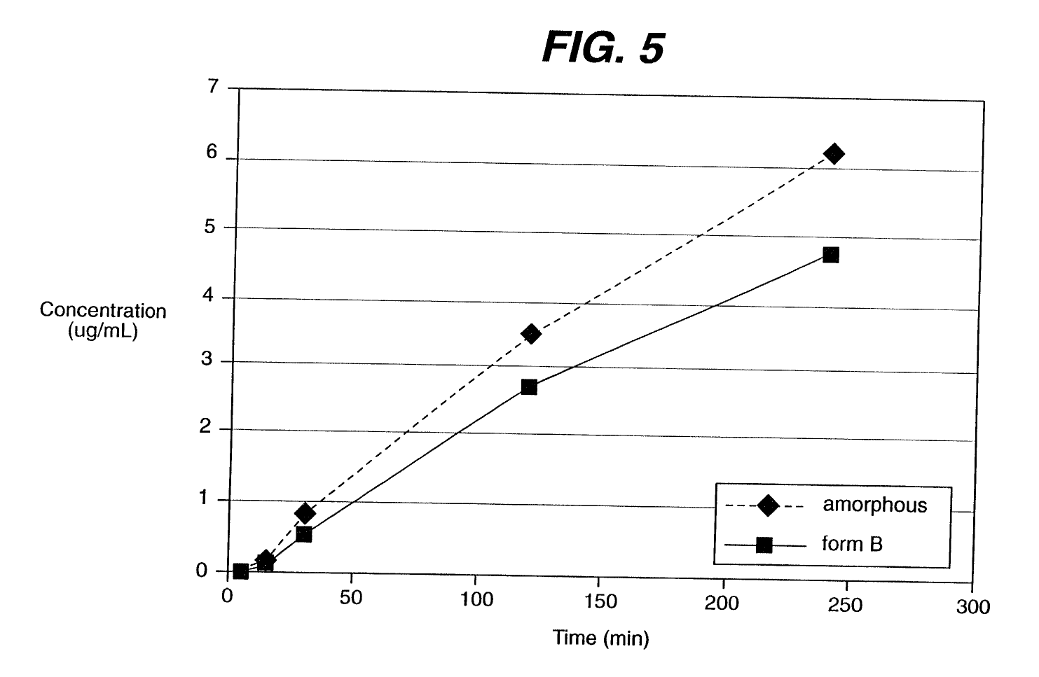

### SUMMARY OF THE INVENTION

The present invention provides an amorphous 5-(4-aminosulfonyl-3-fluorophenyl)-4-cyclohexyl-2-methyloxazole compound, having an improved dissolution rate over crystalline forms. The amorphous compound is characterized by being substantially free of crystals.

The present invention also provides a pharmaceutical composition comprising an amorphous 5-(4-aminosulfonyl-3-fluorophenyl)-4-cyclohexyl-2-methyloxazole compound, substantially free of crystals, and a pharmaceutically acceptable carrier.

The present invention further provides methods for preparing an amorphous 5-(4-aminosulfonyl-3-fluorophenyl)-4-cyclohexyl-2-methyloxazole compound, substantially free of crystals. One embodiment of the method for preparing the amorphous compound, substantially free of crystals, comprises heating the compound and cooling the melt thereof. Another embodiment of the method for preparing the amorphous compound, substantially free of crystals, comprises dissolving the compound in a suitable solvent and recovering by precipitation using a suitable anti-solvent. Still another embodiment of the method for preparing the amorphous compound, substantially free of crystals, comprises dissolving the compound in a suitable solvent and recovering by precipitation using a means for drying.

### DETAILED DESCRIPTION OF THE INVENTION

5-(4-aminosulfonyl-3-fluorophenyl)-4-cyclohexyl-2-methyloxazole compound as described in the Haruta '381 patent, is also referred to by the Chemical Abstracts Society (CAS) Index Name 4-(4-cyclohexyl-2-methyloxazol-5-yl)-2-fluorobenzensulfonamide, having the CAS Registry Number 180200-68-4.

### General Preparative Methods

Amorphous 5-(4-aminosulfonyl-3-fluorophenyl)-4-cyclohexyl-2-methyloxazole compound, substantially free of crystals, can be produced in accordance with the preparative methods described below. Since the methods are illustrations, the invention should not be construed as being limited by the methods and conditions expressed. The preparative methods of the present invention include any other techniques known to those skilled in the art which may be employed to recover the instant amorphous compound. The techniques for preparation of the crystalline 5-(4-aminosulfonyl-3-fluorophenyl)-4-cyclohexyl-2-methyloxazole compound used in the procedures described below are generally within the ordinary skill of the art.

### Method 1

A method for preparing an amorphous 5-(4-aminosulfonyl-3-fluorophenyl)-4-cyclohexyl-2-methyloxazole compound, substantially free of crystals, comprises heating a crystalline compound to form a melt and cooling the melt. In the absence of solvent, the crystalline compound is heated above its melting point and held at that temperature until it is free or substantially free of crystals. The melt can then be allowed to cool at room temperature, or it can be “crash cooled”, as in the case of using an ice bath or forced air cooling.

### Method 2

A method for preparing an amorphous 5-(4-aminosulfonyl-3-fluorophenyl)-4-cyclohexyl-2-methyloxazole compound, substantially free of crystals, comprises dissolving a crystalline compound in a suitable solvent; achieving supersaturation; and recovering the amorphous compound by precipitation using a suitable anti-solvent; hereinafter referred to as “solvent precipitation.”

Suitable solvents for dissolving the crystalline 5-(4-aminosulfonyl-3-fluorophenyl)-4-cyclohexyl-2-methyloxazole compound are solvents selected from the group consisting of tetrahydrofuran, dimethylformamide, acetone, acetone:water (9:1), methyl ethyl ketone, methanol, acetonitrile:water (9:1), ethyl acetate, dichloromethane, ethanol (denatured), acetonitrile, ethanol:water (9:1), 2-propanol and t-butyl alcohol. Preferably, the suitable solvent is selected from the group consisting of tetrahydrofuran, dimethylformamide, acetone, acetone:water (9:1), methyl ethyl ketone, methanol and t-butyl alcohol. More preferably, the suitable solvent is selected from the group consisting of tetrahydrofuran and dimethylformamide. Any of the foregoing suitable solvents may be used alone, as mixtures thereof with water, as mixtures thereof with another of the suitable solvents listed above or as mixtures thereof with water and another of the suitable solvents listed above, wherein the mixtures form a homogeneous phase.

Suitable anti-solvents for recovering an amorphous 5-(4-aminosulfonyl-3-fluorophenyl)-4-cyclohexyl-2-methyloxazole compound by precipitation are ant-solvents selected from the group consisting of water, alkanes, mixtures of alkanes, medium boiling range (from about 40 to about 100° C.) hydrocarbon chain derivatives, ethers and aromatic hydrocarbons. Preferably, the anti-solvent is selected from the group consisting of water, hexane, isopropyl ether, benzene and toluene. More preferably, the anti-solvent is selected from the group consisting of water, hexane and toluene. Most preferably, the anti-solvent is water. The solvent and anti-solvent should be compatible, that is partially miscible; and, preferably, fully miscible.

Solvent precipitation comprises dissolving a crystalline 5-(4-aminosulfonyl-3-fluorophenyl)-4-cyclohexyl-2-methyloxazole compound in a suitable solvent; supersaturating the solvent with the crystalline compound and recovering by precipitation an amorphous 5-(4-aminosulfonyl-3-fluorophenyl)-4-cyclohexyl-2-methyloxazole compound, substantially free of crystals, using an appropriate quantity of a suitable anti-solvent. The anti-solvent is preferably added rapidly, thus causing the amorphous compound to precipitate substantially free of crystals. The precipitated amorphous compound is quickly removed from the solution to avoid the formation of any crystals. As an aid to rapid precipitation, a carrier gas selected from the group consisting of air and nitrogen may be bubbled through the solution; and, preferably, the carrier gas is air. Other aids to rapid precipitation may be used which are deemed to be within the knowledge of those skilled in the art.

The foregoing solvent precipitation method for preparing an amorphous 5-(4-aminosulfonyl-3-fluorophenyl)-4-cyclohexyl-2-methyloxazole compound, substantially free of crystals, may be usefully applied to the method for manufacturing the compound described in the Haruta '381 patent, thus directly obtaining the amorphous compound. The method may comprise adding a suitable solvent to the reaction mixture remaining after 4-cyclohexyl-5-(3-fluorophenyl)-2-methyloxazole or its salt is reacted with chlorosulfonic acid and ammonia to form the crystalline 5-(4-aminosulfonyl-3-fluorophenyl)-4-cyclohexyl-2-methyloxazole compound and recovering an amorphous compound by precipitation using an appropriate quantity of a suitable anti-solvent.

### Method 3

A method for preparing an amorphous 5-(4-aminosulfonyl-3-fluorophenyl)-4-cyclohexyl-2-methyloxazole compound, substantially free of crystals, which comprises dissolving a crystalline compound in a suitable solvent, and recovering by precipitation using a means for drying. The means for drying may be selected from the group consisting of spray-drying, roller-drying and freeze-drying.

Suitable solvents for dissolving the compound are described above in the description of the solvent precipitation method. The solvent and compound are combined in ratios near the saturation point of the compound in the suitable solvent.

The crystalline 5-(4-aminosulfonyl-3-fluorophenyl)-4-cyclohexyl-2-methyloxazole compound has sufficient heat stability, within the temperature range of from about 40° C. to about 100° C., to withstand spray drying. Spray drying systems can be operated by those skilled in the art in a known manner. Closed cycle spray drying systems in which the drying medium is recycled are particularly safe and economical for use in obtaining the product of the present invention.

Spray-drying comprises dissolving a crystalline 5-(4-aminosulfonyl-3-fluorophenyl)-4-cyclohexyl-2-methyloxazole compound in a suitable solvent; heating the solvent to the solvent's boiling point and recovering an amorphous 5-(4-aminosulfonyl-3-fluorophenyl)-4-cyclohexyl-2-methyloxazole compound, substantially free of crystals, by precipitation. The solvated compound is sprayed onto a suitable surface using a drying gas. The solvent is rapidly evaporated, thus causing the amorphous compound to precipitate on the recovery surface substantially free of crystals.

Drying gases for recovering amorphous 5-(4-aminosulfonyl-3-fluorophenyl)-4-cyclohexyl-2-methyloxazole compound by precipitation may be selected from the group consisting of nitrogen, argon, carbon dioxide and air. Preferably, the drying gas is selected from the group consisting of nitrogen, argon and carbon dioxide. Most preferably, the drying gas is nitrogen.

The gas inlet temperature to the spray dryer will be chosen in the range of from about 50° C. to about 100° C. The gas outlet temperature is similarly dependent on the solvent but may, for example, be in the range of from about 40° C. to about 100° C.

Roller-drying comprises dissolving a crystalline 5-(4-aminosulfonyl-3-fluorophenyl)-4-cyclohexyl-2-methyloxazole compound in a suitable solvent; and recovering an amorphous 5-(4-aminosulfonyl-3-fluorophenyl)-4-cyclohexyl-2-methyloxazole compound, substantially free of crystals, by coating the solvated crystalline compound onto a suitable surface using a roller and rapidly evaporating the solvent, thus causing the amorphous compound to precipitate substantially free of crystals onto the recovery surface.

In carrying out the above described spray-drying or roller-drying techniques, it is highly desirable that the boiling point of the solvent used will lie below the coagulation point of the amorphous compound under the conditions employed. In general, the boiling point of the solvent will preferably be below about 80° C., unless reduced pressure is employed thereby allowing the use of higher boiling solvents.

Freeze-drying comprises dissolving a crystalline 5-(4-aminosulfonyl-3-fluorophenyl)-4-cyclohexyl-2-methyloxazole compound in a suitable solvent; and recovering an amorphous 5-(4-aminosulfonyl-3-fluorophenyl)-4-cyclohexyl-2-methyloxazole compound, substantially free of crystals, by rapidly cooling to a temperature at or below the solvent's freezing point, thus causing the amorphous compound to precipitate substantially free of crystals, and the precipitated compound is quickly removed from the solution to avoid the formation of crystals. The temperature at which the recovery will be effected will depend upon the freezing point of the solvent employed such as, for tetrahydrofuran recovery, a temperature of about −108° C. Thus, the choice of preferred solvents for use in freeze-drying is deemed to be within the knowledge of those skilled in the art.

Residual solvent may be present in the final product in varying amounts immediately after evaporation or precipitation. This can be removed, if necessary, by further treatment; preferably, drying under vacuum.

Typically, an amorphous 5-(4-aminosulfonyl-3-fluorophenyl)-4-cyclohexyl-2-methyloxazole compound, substantially free of crystals, is isolated and used as a free base; however, the instant amorphous compound may be isolated and used as a pharmaceutically acceptable salt. For use in medicine, the salts of the amorphous compound of this invention are referred to as non-toxic “pharmaceutically acceptable salts.” Other salts may, however, be useful in the preparation of an amorphous compound according to this invention or of its pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid.

An amorphous 5-(4-aminosulfonyl-3-fluorophenyl)-4-cyclohexyl-2-methyloxazole compound, substantially free of crystals, is a useful COX-2 inhibitor. In particular, the instant amorphous compound inhibits in vitro activity of COX-2 in the nanomolar range.

The amorphous compound of the present invention is useful for treating COX-2 mediated pain and inflammation and inflammatory skin-, eye-, pulmonary- and vascular-related disorders; neuromuscular junction and white matter diseases and other COX-2 mediated cancers, neurodegenerative diseases, cytokine-mediated inflammation and bone resorption disorders in a subject in need thereof. Particularly, the amorphous compound of the present invention is useful for the treatment of COX-2 mediated pain disorders including but not limited to headache, chronic pain and fever. The amorphous compound is useful for the treatment of COX-2 mediated inflammatory disorders including but not limited to rheumatoid arthritis, osteoarthritis, juvenile arthritis, tendinitis and bursitis. Other inflammatory disorders within the scope of the method of treatment include but are not limited to menstrual cramps and preterm labor; liver disease including hepatitis; skin related disorders including psoriasis, eczema, burns and dermatitis; eye related disorders including recovery from ophthalmic surgery, retinitis, conjunctivitis, retinopathies, uveitis, ocular photophobia and acute injury to the eye tissue; inflammatory bowel disease; Crohn's disease; gastritis; irritable bowel disease and ulcerative colitis. Pulmonary inflammation disorders within the scope of the method of treatment include but are not limited to asthma, bronchitis, viral infections and cystic fibrosis. Vascular inflammatory disorders within the scope of the method of treatment include but are not limited to atherosclerosis; migraine headaches; periarteritis nodosa; thyroiditis; aplastic anemia; Hodgkin's disease; sclerodoma; rheumatic fever; type I diabetes; neuromuscular junction disease including myasthenia gravis; white matter disease including multiple sclerosis, sarcoidosis, nephrotic syndrome, Bechet's syndrome and polymyositis; gingivitis; nephritis; hypersensitivity; swelling occurring after injury; myocardial ischemia and the like. Other COX-2 mediated disorders within the scope of the method of treatment using the amorphous compound of the invention include but are not limited to cancers such as colorectal, breast, lung, prostate, bladder and cervix cancer and cancers of the skin; neurodegenerative diseases such as Alzheimer's disease; as an inhibitor for the production of the inflammatory cytokines IL-1 and TNF-a and leukotrienes such as LTB4 and as an agent for bone resorption.

Illustrative of the invention is a pharmaceutical composition made by mixing an amorphous compound described above and a pharmaceutically acceptable carrier. A further illustration of the invention is a process for making a pharmaceutical composition comprising mixing an amorphous compound described above and a pharmaceutically acceptable carrier. The present invention also provides pharmaceutical compositions comprising one or more amorphous compounds of this invention in association with a pharmaceutically acceptable carrier.

An example of the invention is a method for the treatment of a COX-2 mediated disorder in a subject in need thereof comprising administering to the subject a therapeutically effective amount of any of the compounds or pharmaceutical compositions described above. Also included in the invention is the use of an amorphous 5-(4-aminosulfonyl-3-fluorophenyl)-4-cyclohexyl-2-methyloxazole compound, substantially free of crystals, for the preparation of a medicament for treating a COX-2 mediated disorder in a subject in need thereof.

Further exemplifying the invention is a method for the treatment of COX-2 mediated disorders, wherein the therapeutically effective amount of the amorphous compound is from about 0.1 to about 300 mg/kg/day.

In accordance with the methods of the present invention, the individual components of the pharmaceutical compositions described herein can be administered separately at different times during the course of therapy or concurrently in divided or single combination forms. The instant invention is therefore to be understood as embracing all such regimes of simultaneous or alternating treatment and the term “administering” is to be interpreted accordingly.

The utility of the compounds to treat COX-2 mediated disorders can be determined according to the procedures herein. The present invention therefore provides a method for the treatment of COX-2 mediated disorders in a subject in need thereof which comprises administering an amorphous compound as defined herein in a quantity effective to treat COX-2 mediated disorders. An amorphous compound may be administered to a subject in need of treatment by any conventional route of administration including, but not limited to oral, nasal, sublingual, ocular, transdermal, rectal, vaginal and parenteral (i.e. subcutaneous, intramuscular, intradermal, intravenous etc.).

To prepare the pharmaceutical compositions of this invention, an amorphous 5-(4-aminosulfonyl-3-fluorophenyl)-4-cyclohexyl-2-methyloxazole compound, substantially free of crystals, as the active ingredient, is intimately admixed with a pharmaceutical carrier according to conventional pharmaceutical compounding techniques, which carrier may take a wide variety of forms depending of the form of preparation desired for administration (e.g. oral or parenteral).

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein may contain, per unit dosage unit, e.g., tablet, capsule, powder, injection, suppository, teaspoonful and the like, of from about 0.01 mg to 100 mg/kg, preferably from about 0.03-30 mg/kg. More preferably, the dosage form will contain a pharmaceutically acceptable carrier containing between about 0.01 mg and 100 mg, most preferably about 5 to 50 mg, of the amorphous compound. For a 70 kg adult human, an effective amount of the drug is ordinarily supplied at a dosage level of from about 0.01 mg/kg to about 300 mg/kg of body weight per day. Preferably, the range is from about 0.03 to about 100 mg/kg of body weight per day, most preferably, from about 0.03 to about 10 mg/kg of body weight per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

The dosages administered, however, may be varied depending upon the requirement of the subjects, the severity of the condition being treated and the compound being employed. The use of either daily administration or post-periodic dosing may be employed. Advantageously, compounds of the present invention may be administered in a single daily dose or the total daily dosage may be administered in divided doses of two, three or four times daily.

### EXAMPLES

An amorphous 5-(4-aminosulfonyl-3-fluorophenyl)-4-cyclohexyl-2-methyloxazole compound, substantially free of crystals, may be prepared and characterized as described in the following examples which are meant to offer an illustration to aid in the understanding of the invention and to suggest a method of practicing the invention. The examples should not be construed in any way to limit the invention set forth in the claims which follow thereafter. Those skilled in the art may find other methods of practicing the invention which are readily apparent to them. However those methods are deemed to be within the scope of this invention.

### Example 1

In order to obtain an amorphous compound, substantially free of crystals, by the above techniques, it is necessary to employ a starting material of suitable purity; preferably, at least as pure as the final product. Such a starting material is obtained by processes as described in the Haruta '381 patent, or alternatively, is prepared by any conventional method known to one skilled in the art; preferably, by synthesizing a crystalline 5-(4-aminosulfonyl-3-fluorophenyl)-4-cyclohexyl-2-methyloxazole compound using an organic solvent.

### Example 2

X-Ray Powder Diffraction

X-ray powder diffraction analyses were carried out for crystalline compounds having Form A and Form B and the amorphous compound on a Shimadzu XRD-6000 X-ray powder diffractometer using Cu Kα radiation (1.5406 Å). The instrument was equipped with a fine-focus X-ray tube. The tube power was set at 40 kV, 40 mA. The divergence and scattering slits were set at 1° and the receiving slit was set at 0.15 mm. Diffracted radiation was detected by a Nal scintillation detector. A θ-2θ (theta-two theta) continuous scan at 3°/min (0.4 sec/0.02° step) from 4°2θ to 40°2θ was used. A silicon standard was analyzed each day to check the instrument alignment, and an alumina standard was analyzed each day to check the X-ray tube output. Each sample was prepared for analysis by pressing it with a spatula onto a glass or quartz sample holder. For variable temperature runs, the sample was prepared for analysis by pressing it with a glass slide into a variable temperature holder. The powder patterns were collected initially, after heating to various temperatures (110, 145, 150 or 155° C.) for 5 minutes and after cooling to ambient conditions.

An amorphous 5-(4-aminosulfonyl-3-fluorophenyl)-4-cyclohexyl-2-methyloxazole compound, substantially free of crystals, was produced by melting a crystalline 5-(4-aminosulfonyl-3-fluorophenyl)-4-cyclohexyl-2-methyloxazole compound to form a melt and crash cooling the melt to room temperature.

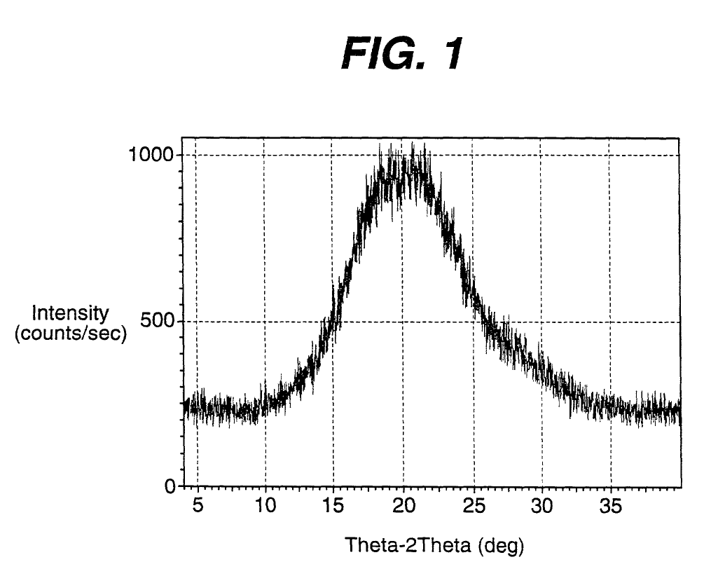

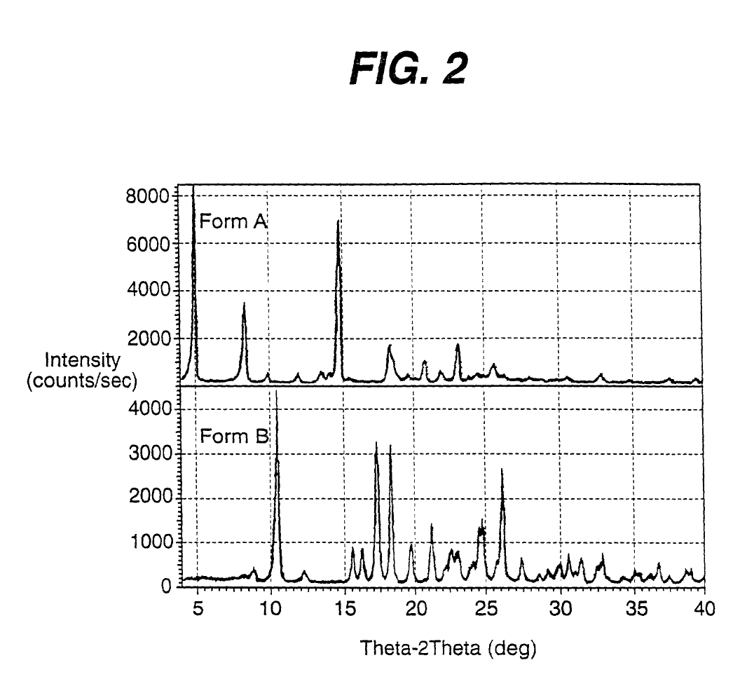

The amorphous form, substantially free of crystals, was characterized by the XRPD pattern as shown in FIG. 1; wherein the novel amorphous compound had a single, smooth, bell-shaped amorphous halo that was distinguished from the XRPD pattern of the crystalline compounds having Form A and Form B as shown in FIG. 2 by the absence of multiple peaks having the interplanar spacings listed in Table 1.

TABLE I
Crystalline Compound Form A and Form B Interplanar Spacings

| Form A (°2θ) | Form B (°2θ) |
|---|---|
| 5.0 | 10.5 |
| 8.3 | 16.2 |
| 13.5 | 17.4 |
| 14.2 | 27.4 |
| 14.9 | |

### Example 3

Amorphous Compound Differential Scanning Calorimetry

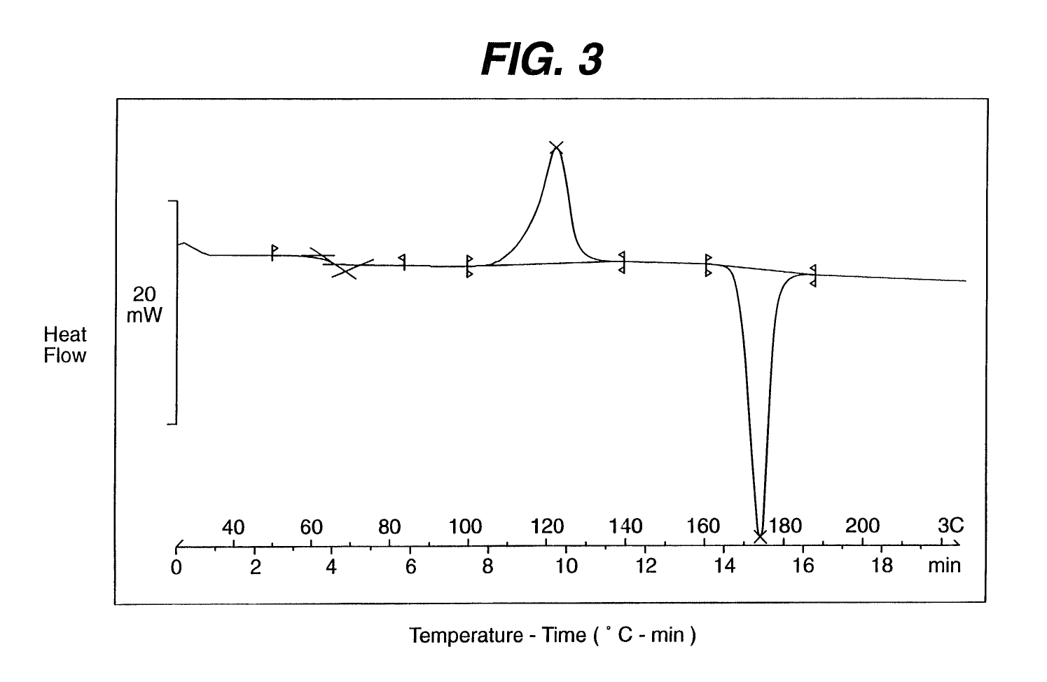

DSC analyses were carried out for the amorphous compound using a Mettler K55143 differential scanning calorimeter. The DSC curve for the amorphous compound shown in FIG. 3 was obtained using a Mettler TC15 TA Controller.

The amorphous compound was produced by heating a 8.8 mg sample at a rate of about 10° C./min from about 25° C. to about 250° C. and allowing the sample to cool to about 25° C. at a rate of about 9° C./min.

The amorphous compound was placed on an unsealed DSC aluminum pan and inserted into the furnace. The amorphous compound sample was then reheated from 25° C. to 250° C. at a rate of 10° C./minute. The thermogram data for the amorphous compound is summarized in Table 2.

TABLE 2
Thermal Data for the Amorphous Compound

| | Onset Temp | Midpoint | Peak Temp | Endpoint |
|---|---|---|---|---|
| Peak | (° C.) | (° C.) | (° C.) | (° C.) |
| Glass transition | 61.99 | 63.00 | — | 65.35 |
| Enthalpic relaxation | — | — | 68.05 | — |
| endotherm | | | | |
| Recrystallization | 113.97 | — | 122.77 | 127.59 |
| exotherm | | | | |
| Melting endotherm | 168.23 | — | 171.32 | 176.51 |

Enthalpic relaxation of the amorphous compound at the glass transition was characterized by a superimposed endotherm at about 60° C. The sample recrystallized without degradation to the amorphous compound after cooling from the melt.

### Example 4

Comparative Dissolution of Crystalline Form B and Amorphous Compound

Dissolution rates for the crystalline compound having Form B and the amorphous compound were determined and compared in two media: water and simulated intestinal fluid (SIF) minus pancreatin.

Initial materials were sieved and material with a particle size between 212 and 425 μm was used for the study. A 150 mg sample was added to 900 mL of fluid in a dissolution bath. A temperature of 37° C. and a paddle speed of 100 rpm were used for the dissolution. Samples were pulled at specific time points (5, 15, 30 120, and 240 min) and filtered through a 0.2 μm nylon filter. Samples were analyzed by HPLC.

In both water (shown in FIG. 6) and SIF (shown in FIG. 7), the crystalline compound having Form B was found to have a significantly slower dissolution rate than the amorphous form. That is, the amorphous compound material dissolved more rapidly.

What is claimed is:

1. An amorphous 5-(4-aminosulfonyl-3-fluorophenyl)-4-cyclohexyl-2-methyloxazole compound.

2. The amorphous compound of claim 1 wherein the amorphous compound is substantially free of crystals.

3. The amorphous compound of claim 1 wherein the amorphous compound has an x-ray powder diffraction pattern characterized by a single, smooth, bell-shaped amorphous halo and the absence of multiple peaks.

4. The amorphous compound of claim 1 wherein the amorphous compound is characterized by an x-ray powder diffraction pattern substantially as shown in FIG. 1.

5. The amorphous compound of claim 1 wherein the amorphous compound is characterized by a differential scanning calorimetry thermogram substantially as shown in FIG. 3.

6. The amorphous compound of claim 1 wherein the amorphous compound upon heating is characterized by a glass transition onset temperature of about 61° C. to about 65.5° C.; a midpoint temperature of about 62° C. to about 64° C.; an enthalpic relaxation endotherm peak temperature of about 67° C. to about 69° C., whereby the enthalpic relaxation endotherm peak is superimposed on the glass transition.

7. The amorphous compound of claim 1 wherein the amorphous compound is characterized by a dissolution rate in water of between about 30% to about 60% greater than the dissolution rate in water of a crystalline 5-(4-aminosulfonyl-3-fluorophenyl)-4-cyclohexyl-2-methyloxazole compound.

8. The amorphous compound of claim 1 wherein the amorphous compound is characterized by a dissolution rate in water of between about 40% to about 50% greater than the crystalline compound.

9. The amorphous compound of claim 1 wherein the amorphous compound is characterized by a dissolution rate in simulated intestinal fluid of between about 20% to about 40% greater than the crystalline compound.

10. The amorphous compound of claim 1 wherein the amorphous compound is characterized by a dissolution rate in simulated intestinal fluid of between about 20% to about 30% greater than the crystalline compound.

11. A method for preparing the amorphous compound of claim 1 which comprises heating the crystalline compound to form a melt and cooling the melt.

12. The method of claim 11 further comprising cooling the melt by allowing the melt to cool.

13. The method of claim 11 further comprising rapidly cooling the melt by placing the melt in an ice bath.

14. The method of claim 11 further comprising heating the crystalline compound at a heating rate of about 10° C. per minute to an onset temperature of between about 169° C. to about 170° C., a peak temperature of about 171° C. and an endset temperature of between about 177° C. to about 178° C. to form the melt and cooling the melt by allowing the melt to cool.

15. The method of claim 14 further comprising rapidly cooling the melt by placing the melt in an ice bath.

16. A method for preparing the amorphous compound of claim 1 which comprises dissolving the crystalline compound in a suitable solvent and recovering the amorphous compound by precipitation using an anti-solvent.

17. A method for preparing the amorphous compound of claim 1 which comprises dissolving the crystalline compound in a suitable solvent and recovering the amorphous compound by precipitation using a means for drying.

18. A pharmaceutical composition made by mixing the amorphous compound of claim 1 and a pharmaceutically acceptable carrier.

19. A medicament prepared by mixing the amorphous compound of claim 1 in a pharmaceutically acceptable formulation.

20. A method for the treatment of cyclooxygenase-2 mediated disorders which comprises administering to a subject in need thereof a therapeutically effective amount of the amorphous compound of claim 1.

21. The method of claim 20 wherein the therapeutically effective amount of the amorphous compound of claim 1 is from about 0.01 mg/Kg/day to about 300 mg/Kg/day.

22. A method for the treatment of cyclooxygenase-2 mediated disorders which comprises administering to a subject in need thereof a therapeutically effective amount of the pharmaceutical composition of claim 18.

23. The method of claim 22 wherein the therapeutically effective amount of the pharmaceutical composition of claim 20 is from about 0.01 mg/Kg/day to about 300 mg/Kg/day.
